(19) United States
(12) Patent Application Publication
(10) Pub. No.: US 2011/0105640 A1
(43) Pub. Date: May 5, 2011

(54) Bone cement having porous fiber reinforcement

(76) Inventors: James J. Mason, Granger, IN (US); Weimin Yue, Warsaw, IN (US)

(21) Appl. No.: 12/925,339

(22) Filed: Oct. 19, 2010

Related U.S. Application Data
(60) Provisional application No. 61/279,371, filed on Oct. 20, 2009.

(51) Int. Cl. A61F 2/28

(52) U.S. Cl. 523/116

(57) ABSTRACT

The bone cement matrix consists of three base component parts, which when mixed form a radiopaque rapidly setting bone cement, namely, a powdered or granular low viscosity polymer component; a liquid reactive or polymerizable monomer component; and a reinforcement component consisting of porous ceramic fibers. The ceramic fibers are porous and have a rough outer surface created by the pores and cavity permeating the fibers. The pores, cavities and rough surface of the fibers themselves, provide the mechanical interface for securely bonding the fibers to the polymer component. During polymerization, the polymer component saturates into the pores and cavities of the ceramic fibers creating a positive interdigitation of the polymer and fibers within the cement matrix.

This invention relates to bone cements, particularly a polymethylmethacrylate (PMMA)-based polymer bone cement matrix with porous ceramic fibers as a reinforcement component and has application to orthopedic implants.

### BACKGROUND AND SUMMARY OF THE INVENTION

Aseptic loosening is a major cause of long-term failure of orthopedic, dental and veterinary implants. Bone cement is an integral component in the installation of many implants, and failure of the bone cement due to fatigue and/or cracking can damage the surrounding bone and lead to a significant increase in pain and discomfort.

Bone cements commonly use polymethylmethacrylate (PMMA), which consists of a powdery bead polymer superficially dissolved by a liquid monomer. Typically, to improve prosthesis fixation, various types of reinforcing phases have been added to PMMA based bone cements. Reinforcing phases, such as, carbon fibers, stainless steel and vitallium wires, titanium sheets, wires and powders, graphite fibers and bio-active glass ceramics have been incorporated into the PMMA matrix of various bone cements with differing degrees of success. For example, U.S. Pat. No. 4,064,566 (Fletcher & Knoell—1977) discloses a bone cement composition comprising of a dispersion of 2 to 12% by weight of high modulus graphite fibers having a diameter from 1 to 50 microns and a length from 0.1 mm to 15 mm within a biocompatible polymer dissolved in a bio-compatible reactive monomer. U.S. Pat. No. 4,963,151 (Ducheyne—1990) discloses a fiber reinforcing material for a two component bone cement, which comprises a bundle of reinforcing fibers held together by an adhesive material which is soluble in the liquid monomer component of the two component bone cement.

Other PMMA based bone cements have used bio-compatible ceramic fibers as a reinforcing phase. Heretofore, ceramic fibers have been preferred for bone cements because these fibers are produced in a sintering process that is designed to gradually reduce the porosity of the fibers, which is typically accompanied by a net shrinkage and overall densification of the fiber. Closing up the pores results in a denser, stronger fiber. Conventional wisdom further suggests that physical defects, such as surface pits, grooves and the relative porosity of the fiber are weaknesses which reduce the fiber's strengthening properties. Consequently, the development of sintered ceramic fibers for PMMA type bone cements has tended toward producing more dense fibers and removing all physical defects from the ceramic fibers themselves.

The bone cement matrix of the present invention consists of three base component parts, which when mixed form a radiopaque rapidly setting bone cement, namely, a powdered or granular low viscosity polymer component; a liquid reactive or polyermizable monomer component; and a reinforcement component consisting of porous ceramic fibers. The bone cement embodying this invention runs contrary to conventional wisdom by employing porous ceramic fibers as the reinforcement component. The ceramic fibers are porous and have a rough outer surface created by the pores and cavity permeating the fibers. The pores, cavities and rough surface of the fibers themselves, provide the mechanical interface for securely bonding the fibers to the polymer component. During polymerization, the polymer component saturates into the pores and cavities of the ceramic fibers creating a positive interdigitation of the polymer and fibers within the cement matrix. While conventional wisdom contends that ceramic fibers with high porosity are undesirable due to reduced density and lessened structural integrity, the porosity of the ceramic fibers provides an improved mechanical interface for the saturation and bonding of the polymer to the fibers. The more porous the fibers and the rougher and more irregular the surface of the fibers, the stronger the interface of the cement matrix. The use of porous ceramic fibers yields a bone cement matrix that is as much as 100 times stronger than conventional bone cements.

Theses and other advantages of the present invention will become apparent from the following description of an embodiment of the invention with reference to the accompanying drawings.

### BRIEF DESCRIPTION OF THE DRAWINGS

The drawings illustrate an embodiment of the present invention, in which:

FIG. 1 is a photograph of the reinforcing ceramic fibers used in the bone cement matrix of the present invention;

FIG. 2 is another photograph of the reinforcing ceramic fibers used in the bone cement matrix of the present invention;

FIG. 3 is a magnified photograph of the sides of the reinforcing ceramic fibers used in the bone cement matrix of the present invention;

FIG. 4 is another magnified photograph of the sides of the reinforcing ceramic fibers used in the bone cement matrix of the present invention;

FIG. 5 is a magnified photograph of an end of a reinforcing ceramic fiber used in the bone cement matrix of the present invention;

FIG. 6 is another magnified photograph of an end and sides of the reinforcing ceramic fiber used in the bone cement matrix of the present invention;

FIG. 7 is a magnified photograph of the surface of the reinforcing ceramic fibers used in the bone cement matrix of the present invention;

FIG. 8 is a magnified photograph of the surface of the reinforcing ceramic fibers used in the bone cement matrix of the present invention;

FIG. 9 is a magnified photograph of the cement matrix of the present invention showing the interdigitation of the reinforcing ceramic fibers and polymer component; and

FIG. 10 is a magnified photograph of the cement matrix of the present invention showing the interdigitation of the reinforcing ceramic fibers and polymer component.

### DESCRIPTION OF THE PREFERRED EMBODIMENT

An embodiment of the bone cement matrix of the present invention consists of three base component parts, which when mixed form a radiopaque rapidly setting bone cement, namely, a powdered or granular low viscosity polymer component; a liquid reactive or polymerizable monomer component; and a reinforcement component consisting of porous ceramic fibers.

The polymer and monomer components of the bone cement matrix of this invention can be based on the acrylic, e.g., (meth)acrylate system of compounds, however, other polymeric compounds may at times be used. In reference to this invention, the terms “(meth)acrylate” and “poly(meth)acrylate” include the monomers and polymers, respectively, of methacrylic acid esters and acrylic acid ethers, and the polymers also include the co-polymers of the same. Generally, the polymer component of the composition can be any methyl(meth)acrylate polymer such as methyl(meth)acrylate homopolymers and copolymers of methyl(meth)acrylate with alpha, beta-ethylenically unsaturated compounds such as vinyl acetate, alkyl (e.g., C₂-C₆) (meth)acrylates and multi-functional acrylic monomers, such as alkylene dimethacrylate and alkylene diacrylates and triacrylates. The monomer component is preferably methyl acrylate or methyl methacrylate although various C₂-C₄ alkyl(meth)acrylates, such as ethyl(meth)acrylate, propyl(meth)acrylate or (n-, or iso-)butyl(meth)acrylate, can also be used. For convenience, the polymer and monomer components of the bone cement matrix of this invention are generally referred to broadly as an acrylic polymer or polymethylmethacrylate (PMMA). These polymer and monomer components are themselves well known and commercially available as is their use in bone cements.

The polymer component also includes a polymerization catalyst, such as benzoyl peroxide (BPO). The monomer component includes a polymerization accelerator, such as N,N-dimethyl-p-toludine (DMPT). When the three matrix components are mixed, the accelerator in the monomer and the catalyst in the polymer component initiate the polymerization of the monomer component, which binds the polymer component together with the ceramic fibers.

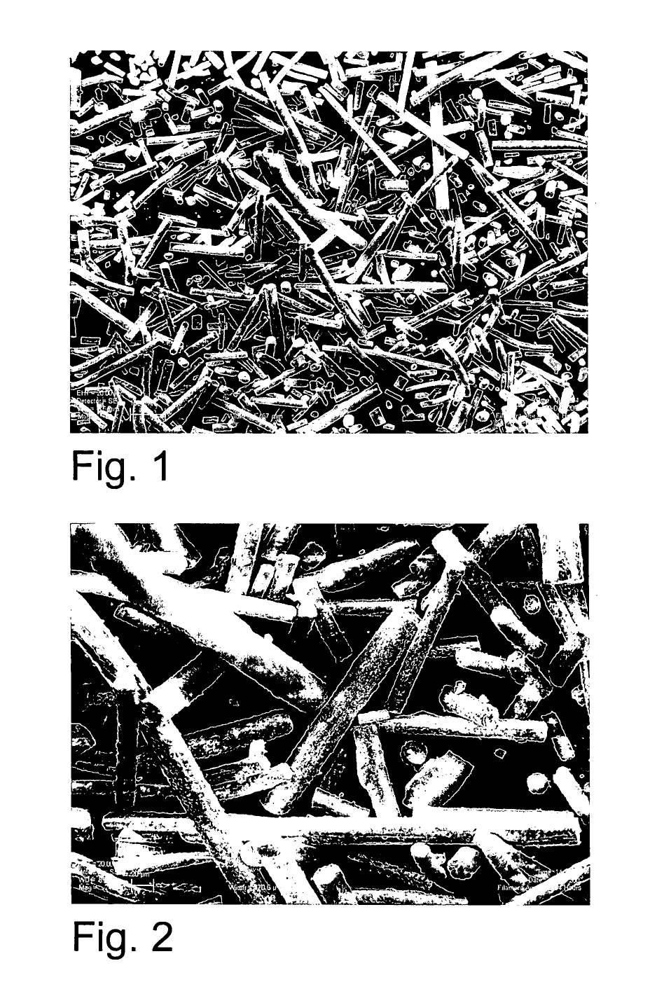

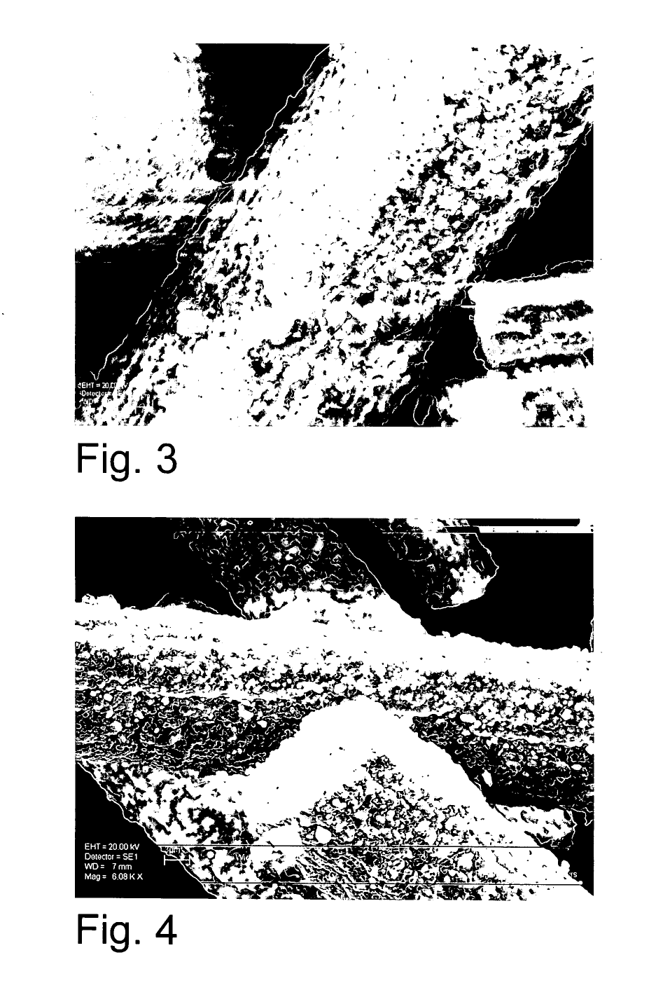

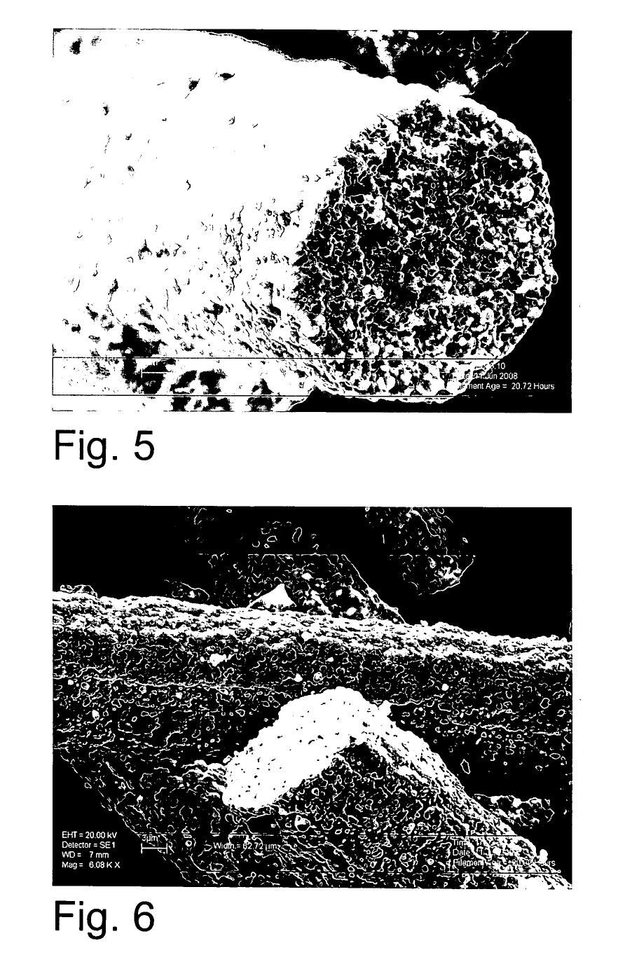

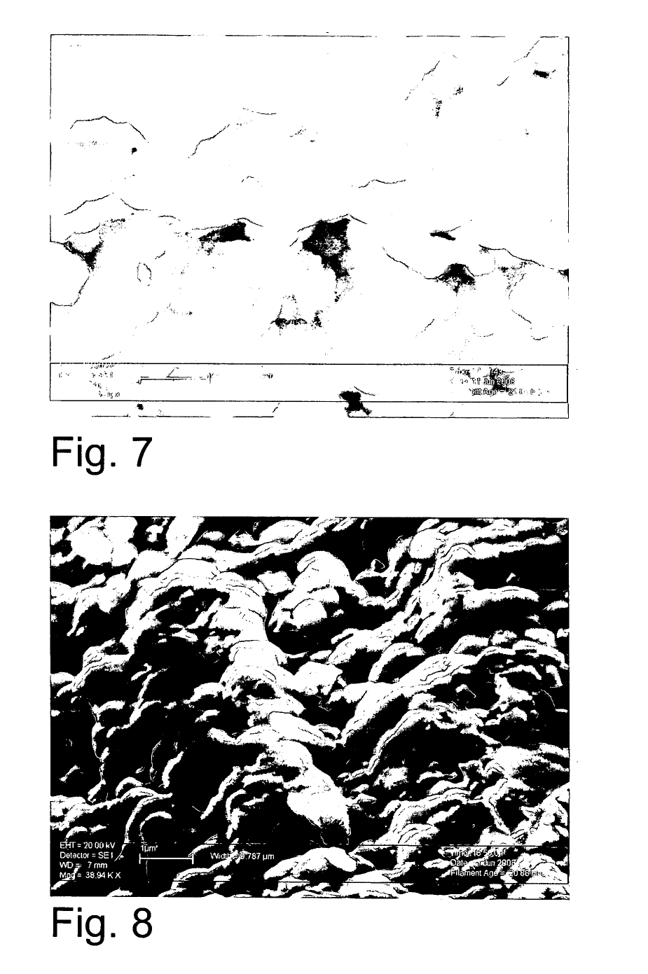

Ideally, the reinforcement component of the cement matrix is held separately as an additive and mixed in proportions with the polymer and monomer components. As shown in FIGS. 1-8, the reinforcement component consists of porous ceramic fibers of zirconium dioxide. The ceramic fibers are of the type developed and produced by Advanced Cerametrics, Inc. of Lambertville, N.J. The ceramic fibers are sintered in a production process that gives the fibers sufficient porosity to allow the interdigitation of the polymer component directly into the fibers themselves. Dimensionally, the ceramic fibers generally have a diameter in the range of 15-20 microns in a continuous form. The fibers can be further milled or chopped down to a length in the range of 15-500 microns so that the fibers can be easily mixed in the cement matrix. As shown in FIG. 1-8, the ceramic fibers are relatively porous and have a rough outer surface created by the pores and cavity permeating the fibers. The pores, cavities and rough surface of the fibers themselves, provide the mechanical interface for securely bonding the fibers to the polymer component. The porosity of the ceramic fibers is generally quantified by the measure of the fibers relative density which is the ratio of the fibers mass to the fibers volume. For example, a solid, non-porous fiber will have a mass equal to its volume and a porous fiber will have a mass less than its volume. The ceramic fibers have a porosity in the range of 5-60 percent, i.e., it mass ranges between 5 and 60 percent of its volume. This range of porosity provides the greatest degree of interdigitation without degrading the structural integrity of the ceramic fibers themselves.

The mixing ratio of the powdered or granule polymer component of PMMA with BPO (as in grams) to the liquid monomer component of MMA with DMPT (as in ml) is in the range of 0.5 to 2. The ratio between PMMA and BPO is in the range of 20-100 to 1. The ratio between MMA to DMPT is in the range of 100-150 to 1. The radio pacifiers, such as barium sulfate could be added. The volume percent of ceramic or polymer fibers used in these fiber reinforced bone cement composites can be anywhere from 2 to 50 volume percent.

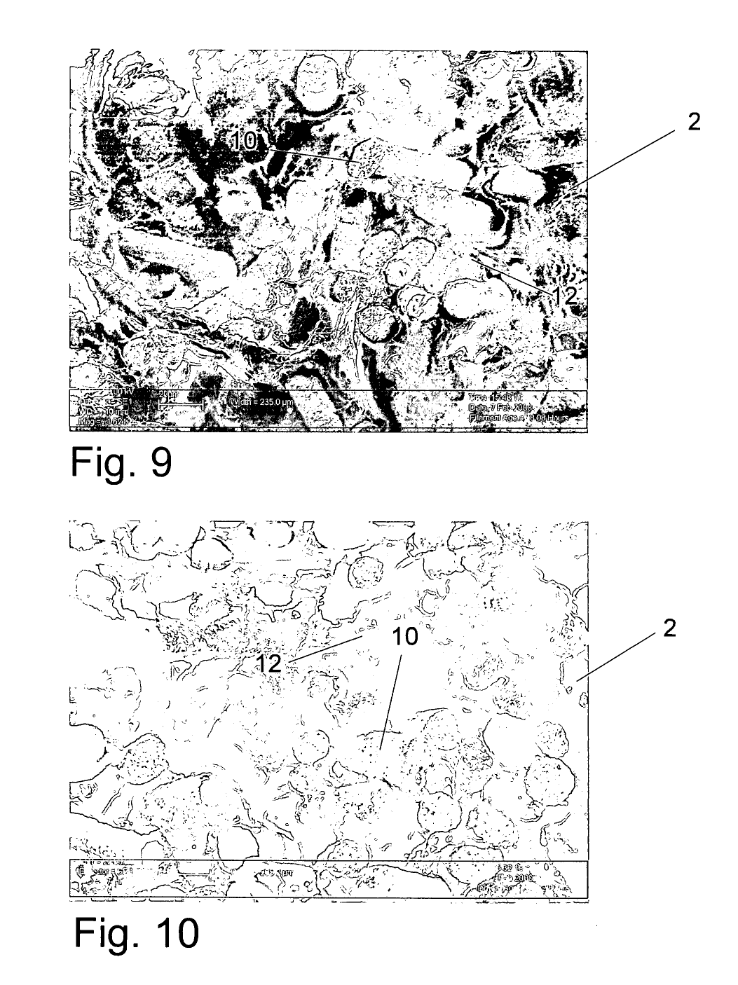

FIGS. 9 and 10 show photographs of the polymerized cement matrix (indicated generally as reference numeral 2) of the present invention showing the interdigitation of the reinforcing ceramic fibers (indicated generally reference numeral 10) and polymer component (indicated generally by the reference 12). During polymerization, polymer component 12 saturates into the pores and cavities of ceramic fibers 10 creating a positive interdigitation of the polymer and fibers within the cement matrix. The porosity of the fibers and the rough, irregular surface of the ceramic fibers provides a strong interface for the cement matrix. The use of porous ceramic fibers yields a bone cement matrix that is as much as 100 times stronger than conventional bone cements.

In alternative embodiments of the present invention, the porous ceramic fibers may be incorporated into the material composition and structure of an typical orthopedic implant, such as hip stems, acetabular plates, fracture rods and plates, and the like. The ceramic fibers may be incorporated directly into the implant's material composition as it is being formed, cast or molded, or may be applied or inlayed on the exterior of the implants. Incorporating the porous ceramic fibers into the implant itself creates an enhanced bonding surface to which the cement matrix will adhere. Again, the polymer components of the cement matrix saturate into the pores and cavities of the porosity, rough, irregular the surface of the exposed ceramic fibers on the surface of the implants forming a solid positive interdigitation between the polymer and fibers in the cement matrix and providing a strong integrated bond directly with the implant.

The embodiments of the present invention herein described and illustrated are not intended to be exhaustive or to limit the invention to the precise form disclosed. They are presented to explain the invention so that others skilled in the art might utilize its teachings. The embodiment of the present invention may be modified within the scope of the following claims.

We claim:

1. A bone cement comprising:
a polymer component;
a monomer component; and
reinforcement fibers held separately as an additive and mixed in proportions with the polymer component and the monomer component, the fibers consist of zirconium dioxide having a porosity defined by the ration of the mass of the fiber to the volume of the fiber within a range of 5 to 60 percent.
